(19) United States
(12) Patent Application Publication
(10) Pub. No.: US 2021/0322297 A9
(43) Pub. Date: Oct. 21, 2021

(54) COSMETIC COMPOSITIONS FOR PROTECTION AGAINST LIGHT-INDUCED DAMAGES

(71) Applicant: GIVAUDAN ITALIA S.P.A, Vimodrone (IT)

(72) Inventors: Giada MARAMALDI, Milano (IT); Martino MENEGHIN, Milano (IT); Stefano TOGNI, Milano (IT)

(21) Appl. No.: 16/970,097

(22) PCT Filed: Feb. 21, 2019
(86) PCT No.: PCT/EP2019/054321

§ 371 Date: Aug. 14, 2020

(30) Foreign Application Priority Data
Feb. 23, 2018 (EP) 18158382.4

(51) Int. Cl. A61K 8/9789; A61Q 17/04

(52) U.S. Cl. CPC A61K 8/9789; A61Q 17/04

(57) ABSTRACT

The invention relates to cosmetic compositions [compositions (C)] comprising an oak extract, a grape seed extract and a green tea extract and to the use of a composition (C) to protect body parts exposed to light against damages. It further relates to a cosmetic method [method (CM)] for protecting body parts exposed to light against damages, said method comprising applying a composition (C) to a body part exposed to light.

This is an application filed under 35 USC 371 based on PCT/EP2019/054321, filed 21.Feb.2019, which in turn is based on EP 18158382.4, 23.Feb.2018. The present application claims the full priority benefit of these applications and herein incorporates by reference the full disclosures of these prior applications.

### TECHNICAL FIELD OF INVENTION

The present invention relates to cosmetic compositions useful to protect body parts exposed to light against damages and to a cosmetic method for protecting body parts against damages due to exposure to light.

### STATE OF THE ART

It is long since known that overexposure to sunlight causes damages to living organisms. In humans, the damages can affect the eyes, the skin, the hair, the scalp and the external mucosae and can have from moderate (e.g. low grade sunburns) to high severity (e.g. skin cancer). Typically, such damages are ascribed to UV rays, i.e. electromagnetic radiations with a wavelength from 10 to 400 nm that are emitted from the sun. UV rays constitute 10% of the total light output of the sun; although most of them (mainly UV-B and UV-C rays, respectively having wavelengths from 280 to 315 nm and from 100 to 280 nm) are filtered by the ozone layer in the earth's atmosphere, the fraction that is not filtered (mainly UV-A rays, having wavelength from 315 to 400 nm) reaches the earth and interacts with living organisms. The depletion of the ozone layer has raised more and more concern, due to the fact that a larger fraction of UV-B and UV-C rays, which are able to induce direct DNA damage and mutagenic/carcinogenic effects reaches the earth. For this reason, the majority of sunscreens for topical applications available on the market contain chemicals able to block UV rays, in particular UV-B rays.

However, in recent years, it has been discovered that not only UV light, but also part of the visible light, namely high-energy light having wavelength from 400 to 495 nm, commonly referred to as “blue light”, is able to cause long-term damages to living organisms, especially due to its ability to induce, similarly to UV-A rays, oxidative stress in the human skin. Oxidative stress results mainly in the carbonylation of proteins in the corneum stratum and also in the extracellular matrix in the dermis and is involved, at least in part, in the generation of xerotic skin in inflammatory skin disorders (like psoriasis vulgaris and atopic dermatitis), skin ageing and skin color changes (references B1 to B8).

The blue light is emitted not only from the sun, but also from many artificial light sources of common and everyday use, such as computer and television screens and from screens of mobile phones. Therefore, there is the need to provide agents able to protect body parts exposed to light, in particular the skin, not only from the adverse effects of UV light, but also from the effects of the blue light.

WO 2017/174718 (Indena S.p.A), published on Oct. 12, 2017, discloses the cosmetic use of a composition comprising an oak extract, a grape seed extract and a green tea extract for the protection against air pollutants, namely from heavy metals. This document neither discloses nor suggests the use of those compositions for the protection against the adverse effects of light, let alone for the prevention of protein carbonylation.

### SUMMARY OF THE INVENTION

The Applicant has now found out that compositions comprising an oak extract, a grape seed extract and a green tea extract are able to protect keratinocytes from carbonylation and, therefore, can be efficiently used to protect body parts exposed to light against damages.

Accordingly, the present invention relates to a cosmetic compositions [composition (C)] comprising an oak extract, a grape seed extract and a green tea extract, to the use of composition (C) to protect body parts exposed to light against damages, in particular against skin damages, and to a cosmetic method [method (CM)] for protecting body parts exposed to light against damages, said method comprising applying composition (C) to a body part exposed to light.

### DESCRIPTION OF THE INVENTION

The present invention relates to a cosmetic composition [compositions (C)] comprising an oak extract [extract (Q)], a grape seed extract [extract (GS)] and a green tea extract [extract (GT)] which is useful to protect body parts exposed to light against damages, in particular against skin damages.

Before disclosing the invention in detail, it is to be pointed out that each publication or patent document cited in the present application is incorporated by reference in its entirety. The citation of any such document is not to be interpreted as an admission that it is prior art with respect to the present invention.

Furthermore, for the avoidance of doubt, within the present application:
unless explicitly stated, the singular forms “a,” “an” and “the” include plural referents. Thus, for example, reference to a “composition (C)” includes any composition comprising the aforementioned extracts.
The term “light” encompasses both sunlight and artificial light, wherein artificial light means any light emitted by artificial sources including, but not limited to, computer and television screens and mobile phones screens, said light comprising radiations having wavelengths from 315 to 400 nm (UV-A) and/or radiations having wavelength from 400 to 495 nm (blue light).
When ranges are indicated, the lowest and highest extremes of the range are included.
The use of round brackets before and after letters, symbols or numbers that identify the compositions and extracts is intended to better distinguish such letters, symbols or numbers from the rest of the text, so those parenthesis could also be omitted.
The expression “body parts exposed to light” means the skin, the scalp, the hair and/or external mucosae; in a preferred embodiment, the body part exposed to light is the skin.

In a compositions (C) according to the invention, oak extract (Q) is preferably an extract characterized by a total polyphenol content ranging between 30% and 60% w/w, more preferably equal to or greater than 45% w/w. Extract (Q) is preferably an aqueous dry extract, i.e. an extract obtained by a process comprising the extraction with water of Quercus robur wood or bark according to methods known in the art.

According to a preferred embodiment, the extract (Q) is obtained from bark.

Extract (Q) can be present in compositions (C) in amounts ranging from 0.01% to 5% w/w, preferably from 0.05% to 1% w/w, more preferably amounts to 0.25% w/w, and even more preferably is 0.1% w/w.

Grape seed extract (GS) is preferably an extract characterized by a total proanthocyanidin content (calculated by the Folin method and expressed as catechins) equal to or greater than 95% w/w and a monomer content (resulting from the sum of catechin and epicatechin expressed as catechin) ranging between 5 and 15% w/w evaluated by the HPLC method, and is more preferably an dry aqueous extract obtained by extraction of grape seeds according to the methods disclosed in WO 2007/017037 A1 (Indena S.p.A., published on Feb. 15, 2007) or in EP 0348781 (TECNOFARMACI S.p.A. and INDENA S.p.A., published on Mar. 1, 1990).

Extract (GS) can be present in compositions (C) in amounts ranging from 0.01% to 5% w/w, preferably from 0.05% to 1% w/w, more preferably amounts to 0.25% w/w, and even more preferably is 0.1% w/w. A particularly suitable grape seed extract suitable for the manufacture of compositions (C) is available on the market from Indena S.p.A. with trademark Enovita®.

Green tea extract (GT) is preferably an extract characterized by a polyphenol content (calculated by the Folin method and expressed as catechins) equal to or greater than 40% w/w, and a catechin content (expressed as epicatechin-3-O-gallate), evaluated by the HPLC method, equal to or greater than 15% w/w, and is more preferably an dry aqueous extract obtained by extraction of green tea dried leaf with water, to provide an extract which is concentrated, homogenized and spray-dried.

According to a preferred embodiment, the green tea extract can be obtained from the leaves.

Green tea extract (GT) may be present in compositions (C) in amounts ranging from 0.01% to 5% w/w, preferably from 0.05% to 1% w/w; more preferably, it amounts to 0.25% w/w, and even more preferably it amounts to 0.1% w/w.

Compositions (C) may contain one or more type of each extract (Q), (GS) and (GT); however, according to a preferred embodiment, compositions (C) comprise only one type of each extract (Q), (GS) and (GT).

Compositions (C) may comprise additional ingredients of plant origin able to exert a protective effect against light. However, according to a preferred embodiment, compositions (C) do not comprise vitamin E or vitamin E derivatives or chromane antioxidants; according to another preferred embodiment, combinations of vitamin E or vitamin E derivatives and chromane antioxidants are excluded from additional ingredients. According to a more preferred embodiment, compositions (C) do not include any such additional ingredients; in other words, compositions (C) are compositions (C-1) comprising only an extract (Q), an extract (GS) and an extract (GT) as active ingredients. In other words, compositions (C-1) consist of an extract (Q), an extract (GS) and an extract (GT).

A preferred composition (C-1) is a composition consisting of an extract (Q), an extract (GS), extract (GT) in the same weight amounts; such composition (C-1) is already available on the market from Indena S.p.A. with tradename Vitachelox™.

Experiments carried out by the Applicant have demonstrated that compositions (C) are able to significantly reduce protein oxidative damage (carbonylation) of proteins induced by light, in particular blue light, in human keratinocytes; therefore, compositions (C) can be effectively used for the protection of the body from light and for the prevention of damages induced by light, in particular damages induced by UV-A and blue light radiations, especially damages induced by blue light radiations, including, but not limited to, xerotic skin, inflammatory skin disorders (like psoriasis vulgaris and atopic dermatitis), skin ageing and skin color changes.

Thus, compositions (C) can be applied topically to body parts exposed to light; typically, compositions (C) are applied in combination with cosmetically acceptable excipients [excipients (E)]. Therefore, according to a further embodiment, the present invention relates to a method [method (M)] for preparing a cosmetic formulation [formulation (F)] comprising mixing a composition (C) with one or more excipients (E) and to the non-therapeutic or cosmetic use of compositions (C) and formulations (F) for the protection against light damages. The present invention further relates to a cosmetic method [method (CM)] comprising applying a composition (C) or a formulation (F) to a body part exposed to light.

Formulations (F) can be obtained by conventional techniques as described, for example, in “Remington's Pharmaceutical Sciences”, Mack Publishing Co., N.Y., USA, 14th Edition, and in “Handbook of Pharmaceutical Excipients” by R. C. Rowe et al., Pharmaceutical Press, 7th Edition.

Examples of formulations (F) are emulsions, gels, foundations, lipsticks and ointments. A person skilled in the art shall be able to select excipients (E) among those known in the art of cosmetics according to the specific formulations (F) to be obtained. Excipients (E) are also disclosed, for example in “International Cosmetic Ingredient Dictionary and Handbook”, published by Personal Care Products Council, 16th Edition, 2016.

The amount of a composition (C) in a formulation (F) typically ranges from 0.001% w/w to 10% w/w, preferably from 0.001% w/w to 1% w/w, more preferably from 0.001% w/w to 0.5% w/w, even more preferably from 0.01% w/w to 0.5% w/w with respect to the overall weight of the formulation (F).

The invention is described in greater detail in the experimental section below.

### EXPERIMENTAL SECTION

Evaluation of the Protective Efficacy of a Composition (C-1) against Blue Light (460 nm)—Induced Protein Oxidative Damage (Carbonylation)

Outline of the Experimental Procedure

Human keratinocytes (HaCAT) were irradiated in the presence or absence of a composition (C-1) comprising equal weight amounts of extracts (Q), (GS) and (GT) (Vitakelox™) using an irradiation system available from OxiProteomics®. A solution of N-acetyl-cysteine was used as positive control of protection. Just after irradiation, proteins were extracted from the cells and analyzed. Carbonylated proteins were labeled with specific functionalized fluorescent probes and the resulting samples were resolved by high-resolution electrophoresis. Total proteins were post-stained with Sypro™ Ruby protein gel stain. A Carbonyl (protein damage) Score was obtained for each sample after normalization of the fluorescent signal for carbonylated proteins by total proteins. A significant increase in protein oxidative damage was observed upon blue light irradiation. This increase was prevented in the presence of composition (C-1) at two different concentrations (0.01% and 0.005% w/v) and with two times of treatment (6 hours and 24 hours).

Detailed Experimental Procedure

a) Cell Culture and Experimental Design

HaCaT cells were cultured in calcium-free DMEM (Dulbecco's Modified Eagle Medium), with 10% SVF (stromal vascular fraction), at 37° C. and humid atmosphere, supplemented with 5% CO₂. Cells were seeded (500.000 cells/well) at day 0 (D0) in multi-well plates containing culture medium and distributed in 8 experimental groups as depicted in Table 1.

TABLE 1

| | | | Sampling |
|---|---|---|---|
| Group | Reference | Replicates | (time) |
| G-1 | Control (not irradiated) | 3 | Day 2 |
| G-2 | Stressed* | 3 | Day 2 |
| G-3 | Composition (C-1), 0.01%, 6 hours | 3 | Day 2 |
| G-4 | Composition (C-1), 0.01%, 24 hours | 3 | Day 2 |
| G-5 | Composition (C-1), 0.05%, 6 hours | 3 | Day 2 |
| G-6 | Composition (C-1), 0.05%, 24 hours | 3 | Day 2 |
| G-7 | Internal reference**, 6 hours | 3 | Day 2 |
| G-8 | Internal reference**, 24 hours | 3 | Day 2 |

*stress was induced by irradiation (see point c) below.
**N-acetyl-cysteine

b) Application of Composition (C-1)

On day 1 (D1), two solutions of composition (C-1) having two different concentrations (0.01% and 0.005%) were prepared by solubilisation of the composition in the culture medium. Experimental groups 4 and 6 were incubated for 24 hours (t2) with the solutions prior to irradiation. At day 2 (D2), experimental groups 3 and 5 were incubated for 6 hours (t1) prior to blue light irradiation. Groups 7 and 8 were incubated with a solution of NAC (N-acetyl-cysteine) for 6 and 24 hours.

c) Blue Light Irradiation and Sampling

On day 2 (D2), the cells were washed and a stress was induced by irradiation with Blue Light (LED source, emission peak at λ=460 nm), using an OxiProteomics® irradiation system. Group 1 was not irradiated. Just after irradiation, cells were collected, snap-frozen and stored at −80° C. for the analysis.

Analysis

a) Carbonyl View (In-Situ Visualization)

After treatments with composition (C-1) and irradiation, the cells were fixed by using a mix of 95% ethanol and 5% acetic acid. Carbonylated proteins were labelled by using a specific functionalized fluorescent probe. The total proteins were labelled using Cy3 NHS (N-hydroxysulfosuccinimide) fluorescent probe, which is routinely used in proteomics studies (see, for instance J Chromatogr B Analyt Technol Biomed Life Sci. 2011 May 15; 879(17-18):1439-43). Images were collected using an epifluorescence microscope and analysed with the software imageJ (Rasband, W. S., ImageJ, U.S. National Institutes of Health, Bethesda, Md., USA, http://imagej.nih.gov/ij/, 1997-2014). The comparison between the different conditions was achieved with strictly identical exposure time, focus and resolution.

b) Carbonyl Score Analysis

Proteins were extracted from cells, quantified by the Bradford method and split into equal amounts for analyses. The carbonylated proteins were labelled with specific functionalized fluorescent probes and the samples were resolved by high-resolution electrophoresis separation. Total proteins were post-stained with SyproRuby™ protein gel stain. Image acquisition for carbonylated and total proteins was performed using the Ettan® DIGE imager (GE Healthcare). Image processing and analysis was performed using ImageJ (Rasband, W. S., ImageJ, U.S. National Institutes of Health, Bethesda, Md., USA, http://imagej.nih.gov/ij/, 1997-B014). Statistical analyses were accomplished using GraphPad Software (La Jolla, Calif., USA).

Results

Carbonyl View

The in-situ oxidation level (carbonylation) was represented as the superposition of oxidative specific signal (red) and total protein signal (green). Differences in the specific oxidative protein patters were observed for the condition “control” and “irradiated”. The concentration of fluorescent probes was in excess ensuring an exhaustive labelling of proteins. Image collection was conducted in strictly identical conditions (focus, zoom, resolution, exposure time). The presence of composition (C-1) decreased blue light induced oxidation.

Carbonyl Score

After extraction and solubilisation, proteins in samples were quantified by the Bradford method using calibrated BSA (bovine serum albumin) as standard (Bradford M. Anal. Biochem., 72, 248, 1976).

After labelling carbonylated proteins with fluorescent probes, the proteins were resolved onto 4-20% gradient SDS-PAGE. Proteins were fixed to the gel and the carbonylated proteins were evidenced by fluorescence scanning. Total proteins were post-stained with SyproRuby.

Densitometric analysis of protein bands was performed using ImageJ analysis software (NIH, USA).

Quantification was obtained from each sample, both for carbonylated and total proteins. Carbonylated protein signal was normalized by total protein signal for each sample in order to obtain the Carbonyl Score.

$$Carbonyl Score ( sample X ) = \frac{carbonylated prot . fluorescent signal ( sample X )}{total prot . fluorescent signal ( sample X )}$$

Carbonyl Score values were quantified for each sample and the average values were also calculated for each experimental condition taking into consideration the replicates; the results are reported in Table 2 here below.

TABLE 2
Carbonyl Score

| Group (G) and | | | | |
|---|---|---|---|---|
| replicate (R) | Oxidized | Total | Carbonyl | |
| number | proteins | Proteins | Score | Condition |
| G-1(R1) | 11833.33 | 21509.91 | 0.550 | Control |
| G-1(R2) | 11082.24 | 18175.04 | 0.610 | |
| G-1(R3) | 11995.13 | 19558.61 | 0.613 | |
| G-2 (R1) | 13293.93 | 16795.54 | 0.792 | Stressed |
| G-2 (R2) | 14562.57 | 18606.68 | 0.783 | |
| G-2 (R3) | 18147.06 | 20767.23 | 0.874 | |
| G-3 (R1) | 15145.65 | 17046.06 | 0.889 | Composition (C-1), |
| G-3 (R2) | 12443.48 | 16265.65 | 0.765 | 0.01% 6 h |
| G-3 (R3) | 12097.48 | 16026.06 | 0.755 | |
| G-4 (R1) | 13306.06 | 17444.77 | 0.763 | Composition (C-1), |
| G-4 (R2) | 11831.18 | 16069.77 | 0.736 | 0.01% 24 h |
| G-4 (R3) | 11839.48 | 15942.77 | 0.743 | |
| G-5 (R1) | 10470.77 | 15506.89 | 0.675 | Composition (C-1), |
| G-5 (R2) | 9276.77 | 15245.48 | 0.608 | 0.005% 6 h |
| G-5 (R3) | 10370.36 | 16905.18 | 0.613 | |
| G-6 (R1)* | 14599.31 | 16357.18 | 0.893 | Composition (C-1), |
| G-6 (R2) | 10036.23 | 16145.41 | 0.622 | 0.005% 24 h |
| G-6 (R3) | 10445.06 | 15188.06 | 0.688 | |
| G-7 (R1) | 12101.36 | 16480.48 | 0.734 | NAC 6 h |
| G-7 (R2) | 11379.65 | 15343.94 | 0.742 | |
| G-7 (R3) | 10415.94 | 15861.06 | 0.657 | |
| G-8 (R1) | 9126.82 | 14273.53 | 0.639 | NAC 24 h |
| G-8 (R2) | 11501.94 | 16425.23 | 0.700 | |
| G-8 (R3) | 10859.53 | 17197.65 | 0.631 | |

A significant increase in oxidized proteins was observed upon irradiation with blue light [group (G-2)].

Composition (C-1) showed a protective effect against blue light-induced oxidative damage on proteins at both concentrations (0.01% and 0.005% w/v) after 24 h of incubation. A statistically significant protection (p<0.01) against blue light-induced damage was observed for composition (C-1) at 0.005% (w/v) at both time points of incubation (6 h and 24 h).

### BIBLIOGRAPHY

B1) Fujita, H. et al., A simple and non-invasive visualization for assessment of carbonylated protein in the stratum corneum, Skin Research and Technology 2007; 13: 84-90.

B2) Mizutani, T. et al., Carbonylated proteins exposed to UVA and to blue light generate reactive oxygen species through a type I photosensitizing reaction, Journal of Dermatological Science 84 (2016) 314-321.

B3) Sander, C. S., Photoaging is Associated with Protein Oxidation in Human Skin In Vivo, The Journal of Investigative Dermatology, Vol. 118, No. 4 April 2002.

B4) IWAI, I., Increased carbonyl protein level in the stratum corneum of inflammatory skin disorders: A non-invasive approach, Journal of Dermatology 2010; 37: 693-6.

B5) Nakashima, Y., Blue light-induced oxidative stress in live skin, Free Radical Biology and Medicine 108 (2017) 300-310.

B6) Davies M. J., Protein oxidation and peroxidation, Biochem. J. (2016) 473, 805-825.

B7) Kobayashi, Y., Increased carbonyl protein levels in the stratum corneum of the face during winter, International Journal of Cosmetic Science, 2008,30,35-40.

B8) Lodovici, M., Oxidative Stress and Air Pollution Exposure, Journal of Toxicology, Vol. 2011, Hindawi Publishing Corporation.

1. A method of protecting body parts from damage induced by light radiation, the method comprising the step of:
application of a cosmetic composition comprising an oak extract, a grape seed extract and a green tea extract to body parts exposed to the light radiation.

2. The method of claim 1, wherein the light radiation has a wavelength of from 315 to 400 nm (UV-A) and/or a wavelength from 400 to 495 nm.

3. The method of claim 2, wherein the light radiation has a wavelength of from 400 to 495 nm.

4. The method of claim 1, wherein the body part is the skin, the scalp, the hair and/or external mucosae.

5. The method of claim 4, wherein the body part is the skin.

6. The method of claim 1, wherein the oak extract has a total polyphenol content ranging from 30% to 60% w/w.

7. The method of claim 1, wherein the oak extract is present in the cosmetic composition at from 0.01% to 5% w/w.

8. The method of claim 1, wherein the grape seed extract has a total proanthocyanidin content, as calculated by the Folin method and expressed as as catechins, equal to or greater than 95% w/w, and has a monomer content, resulting from the sum of catechin and epicatechin expressed as catechin, ranging from 5% to 15% w/w.

9. The method of claim 1, wherein the grape seed extract is present in the cosmetic composition at from 0.01% to 5% w/w.

10. The method of claim 1, wherein the green tea extract has a polyphenol content, as calculated by the Folin method and expressed as catechins, equal to or greater than 40% w/w and has a catechin content, as expressed as epicatechin-3-O-gallate, equal to or greater than 15% w/w.

11. The method of claim 1, wherein the green tea extract is present in the cosmetic composition at from 0.01% to 5% w/w.

12. The method of claim 1, wherein the damage induced by light radiation is selected from: xerotic skin in inflammatory skin disorders, skin ageing and skin color changes.

13. The method of claim 1, wherein the cosmetic composition further comprises one or more cosmetically acceptable excipients.

14. The method of claim 11, wherein the green tea extract is present in the cosmetic composition at from 0.05% to 1% w/w.

15. The method of claim 1, wherein the green tea extract is present in the cosmetic composition at 0.25% w/w.

16. The method of claim 1, wherein the green tea extract is present in the cosmetic composition at 0.1% w/w.
